(19) United States
(12) Patent Application Publication
(10) Pub. No.: US 2002/0168643 A1
(43) Pub. Date: Nov. 14, 2002

(54) Devices and methods for simultaneous separation and purification of molecular samples

(76) Inventors: Jamey M. Wierzbowski, Waltham, MA (US); Alan Dupes, Plymouth, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/851,540

(22) Filed: May 8, 2001

(51) Int. Cl. C12Q 1/68; C12M 1/34; G01N 33/559; G01N 33/561

(52) U.S. Cl. 435/6; 435/287.2; 436/516; 204/461

(57) ABSTRACT

This invention provides devices and methods for the simultaneous separation and purification of molecular samples by electrophoresis. In a preferred embodiment, the separation and purification of molecular samples is provided in a two-dimensional array. In a further embodiment of the invention, gas removal passageways are provided to remove gas produced by electrodes that are immersed in aqueous solution.

### FIELD OF THE INVENTION

This invention relates to devices and methods for the separation and purification of molecular samples, and more particularly to electrophoretic devices and methods for the separation and purification of multiple molecular samples.

### BACKGROUND OF THE INVENTION

The field of molecular biology comprises a variety of techniques for the separation, isolation, and purification of molecular samples. Electrophoresis is a widely used technique for separating biological molecules, such as DNA fragments or proteins, from a mixture of similar molecules. Electrophoresis is also useful to separate organic and inorganic compounds.

Typically, electrophoresis involves applying a sample to an electrophoretic medium and applying an electric current to cause the sample to migrate through the medium. The components of the sample migrate at different rates due to their differences in size and charge. As a result, the components of the sample are separated from each other in the electrophoretic medium, and these separated components can be isolated and purified for subsequent manipulation or analysis.

The prior art contains numerous devices and methods that are available for purifying molecules and fragments separated on agarose and acrylamide gels. However, the prior art generally requires cutting a region of interest out of a gel to isolate the desired molecule or molecular fragment. In addition, before the region of interest is cut out, the gel is often visualized with ultraviolet (UV) radiation which can damage the molecule or fragment and restrict its use in later processes. Furthermore, after a gel slice has been obtained, additional manipulation is required to purify a desired molecule, for example, by processing the gel slice using an elution device.

Known devices and methods are time consuming, laborious, and expensive, particularly if multiple samples are to be processed. For example, hierarchical shotgun sequencing of DNA requires the production of shotgun plasmid sequencing libraries sub-cloned from larger insert clones, such as from bacterial artificial chromosomes. In large sequencing operations, this typically requires size fractionating hundreds of sheared clones. In addition to time and cost concerns, known devices and methods are not adapted for reproducibly isolating DNA fragments of a preselected size range from large numbers of size fractionated samples.

In order to efficiently and reproducibly separate and isolate specific biological molecules from molecular samples, it is highly desirable to provide devices and methods that simultaneously and uniformly process multiple samples. Accordingly, there is a need in the art for high-throughput devices and methods that simultaneously separate and purify molecular samples reproducibly. Such a device and method is provided herein.

### SUMMARY OF THE INVENTION

The present invention is directed to devices and methods for conducting simultaneous separation and purification of a plurality of molecular samples using a two-dimensional sample array. According to the invention, multiple samples are separated and purified in a single procedure using a single device. Target components are separated in an electrophoretic medium and are directly electrophoresed into a collection unit that is attached to the electrophoretic medium, without needing to manipulate the molecular sample between separation and purification steps. Using the invention contained herein, it is possible reproducibly to separate and purify components from a plurality of samples in a single analysis using a two-dimensional sample array.

The invention provides electrophoretic devices and methods that are useful to process multiple samples simultaneously in a two-dimensional array of wells. In order to process multiple samples at the same time with identical electrophoretic migration rates, a uniform electric current is applied across the array of wells. A uniform migration rate in all of the wells allows molecules of the same size to be simultaneously separated and purified in the different wells. Devices and methods of the invention provide for gas removal passageways that remove gas from buffer in which the electrodes are located. According to the invention, a uniform electric current is maintained during electrophoresis by removing gas bubbles that develop at the electrodes, thereby reducing the accumulation of gas bubbles on the two-dimensional well array. An excessive accumulation of gas bubbles would disrupt the uniformity of the electric current across the two-dimensional well array.

In one aspect, the invention relates to a device and apparatus for simultaneously separating and purifying a plurality of molecular samples by electrophoresis. In one embodiment, the device has a housing with an upper surface and lower surface, a two-dimensional array of a plurality of sample wells that are in fluidic communication with the upper and lower surface, and one or more gas removal passageways that are in fluidic communication with the lower surface. In another embodiment, the apparatus has a housing with an upper and lower surface, a plurality of sample wells in fluidic communication with the upper and lower surfaces, one or more gas removal passageways on the lower surface, and an electrode located substantially under said passageways. Gas removal passageways preferably connect the lower surface of the housing to the upper surface of the housing. In operation, gas bubbles from an electrode in an electrophoretic buffer, are captured by a gas removal passageway and thereby removed from the lower surface of the housing. Alternatively, gas removal passageways extend along the lower surface of the housing and are adapted to direct gas bubbles away from the electrodes towards the sides of the housing without interfering with the uniformity of the current through a sample well. In a further aspect, the invention provides a collection unit that is adapted to fit on a two dimensional sample block of the invention. Preferably, the collection unit and sample block include a register to ensure that the collection unit aligns correctly with the sample block, such that collection elements of the collection unit are coaxial with sample wells of the sample block. In addition, a collection unit may attach to the sample block so that they remain aligned during operation.

In another aspect, the invention relates to an electrode for generating a uniform current across an array of electrophoretic wells. The electrode preferably has an electrically conducting plate and an electrically insulating plate, both having upper and lower surfaces. The lower surface of the electrically insulating plate includes a plurality of electrode pins. The lower surface of the insulating plate is attached to the upper surface of the conducting plate. Each electrode pin is electrically connected to the conducting plate and the upper surface of the insulating plate. In one embodiment, the electrode pins are arranged in a two-dimensional array. In one preferred embodiment, the preferred electrode includes 117 electrode pins.

In a further aspect, the present invention broadly relates to methods for simultaneously separating and purifying molecular samples in a two-dimensional array. According to the method of this invention, molecular samples are loaded onto an electrophoretic medium and an electric field is applied across the medium to induce the molecular samples to migrate through the medium. A collection unit is attached to the medium to collect a target component of the sample when the molecular samples have reached a predetermined position in the medium. A tracking agent can be used to monitor the position of the molecular sample as it migrates through the medium. In a preferred aspect, the invention provides a method for simultaneously preparing multiple samples in parallel. Preferably, a uniform current is applied across the two-dimensional array of wells to provide uniform electrophoretic migration of the samples through the wells. An electric current is preferably applied via an upper electrophoretic buffer in electrical connection with the upper surface of the sample wells and a lower electrophoretic buffer in electrical connection with the lower surface of the sample wells. The upper and lower electrophoretic buffers are connected to a power supply to apply an electric field to the sample wells. In a further aspect of the invention, passageways are provided to remove gas produced by the electrodes that are immersed in the upper and lower electrophoretic buffers.

### BRIEF DESCRIPTION OF THE DRAWINGS

The invention is pointed out with particularity in the appended claims. The advantages of the invention described above, as well as further advantages of the invention, may be better understood by reference to the following detailed description taken in conjunction with the accompanying drawings, in which:

FIG. 1 is an exploded three-dimensional diagram of an embodiment of an electrophoretic elution device constructed in accordance with the invention.

FIG. 2 is a cross-sectional view diagram through line A-A′ of the embodiment of an electrophoretic elution device shown in FIG. 1.

FIGS. 3 a, b, c are the top and two side diagrams, respectively, of the embodiment of the upper buffer chamber shown in FIG. 1.

FIGS. 4 a, b, c are the top and two side diagrams respectively of the embodiment of a sample block shown in FIG. 1.

FIGS. 5 a, b, c are the top and two side diagrams respectively of the embodiment of the bottom electrode unit shown in FIG. 1.

### DETAILED DESCRIPTION OF THE INVENTION

The devices and methods of the invention provide for the simultaneous electrophoretic separation and purification of multiple molecular samples in a two-dimensional array. The invention is particularly useful for separating and purifying biological molecules, including, but not limited to, nucleic acids, DNA fragments, RNA fragments, peptides, polypetides, proteins, and biopolymers. In one embodiment, the electrophoretic medium is an agarose, acrylamide, or polyacrylamide gel medium, but any electrophoretic medium may be used.

The present invention provides the advantages of separating and purifying multiple samples simultaneously. The invention further provides the advantages of providing an electrode that delivers uniform current across a two-dimensional array of wells. Additionally, the invention provides the advantage of providing passageways or conduits for the removal of gas bubbles produced by electrodes immersed in aqueous solution.

The devices and methods of the invention are especially useful for separating a large number of biological samples. Devices and methods of the invention have a wide range of uses, including, but not limited to, purifying protein samples, isolating particular fragments from a DNA restriction endonuclease digest, preparing a size-fractionated genomic DNA, and separating sheared BAC (bacterial artificial chromosome) DNA based on size for sub-cloning genomic DNA into a high-copy plasmid vector. Additionally, devices and methods of the invention are useful for purifying polymerase chain reaction (PCR) products, including removing unincorporated deoxynucleotide triphosphates (dNTP's) as well as products of undesired size. The present invention is useful for collecting any size range of fragments. An important feature of the present invention is the ability to purify multiple samples simultaneously.

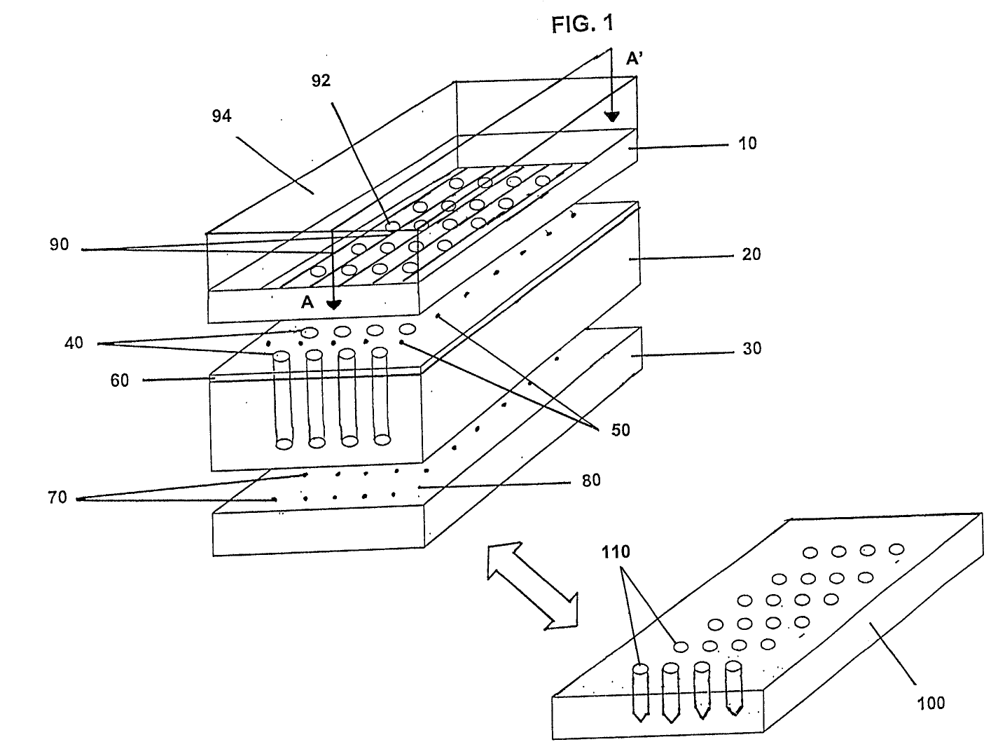

FIG. 1 shows an embodiment of the invention that includes an upper buffer chamber 10 containing a plurality of platinum electrode wires 90, a sample block 20 and a bottom electrode unit 30. In this embodiment, the upper buffer chamber 10 contains a plurality of sample well openings 92. The sample block 20 includes a plurality of sample wells 40 shaped as tubular columns extending through the sample block 20 and positioned to match the well openings 92 of the upper buffer chamber 10. Capillary gas removal passageways 50 extend through the sample block 20, and a sealing material 60 seals the join between the upper buffer chamber 10 and the sample block 20 but permits liquid and current to pass from the upper buffer chamber 20 through to the sample wells 40 of the sample block 20. The bottom electrode unit 30 contains, in one embodiment, a copper conducting board 80 with an array of platinum electrode pins 70 attached thereto and pointed toward the sample wells 40 of the sample block 20.

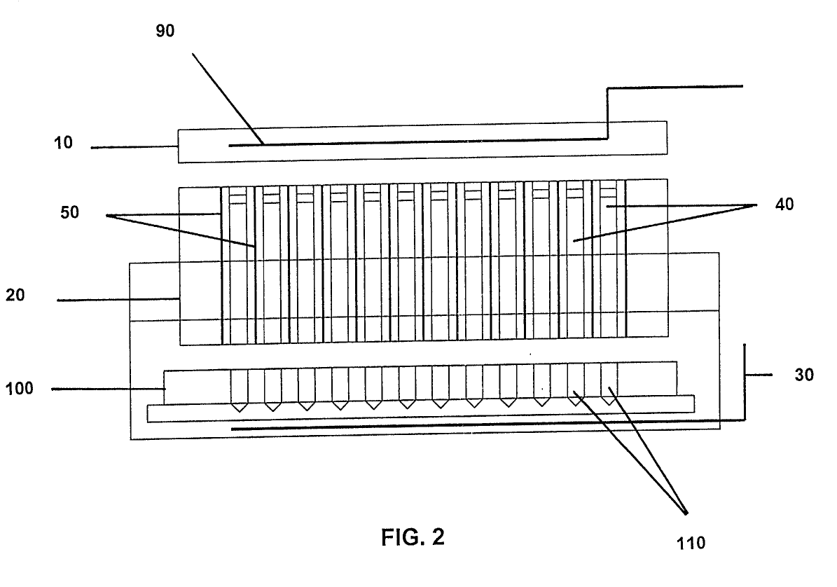

Further, FIGS. 1 and 2 show a collection unit 100 that is used to collect sample fractions from the sample wells. The collection unit can be connected to the bottom of sample block 20 so that components of the molecular samples in the sample wells 40 can be electrophoresed into the collection unit. A collection unit preferably includes a plurality of collection chambers 110. Each collection chamber 110 is preferably coaxial with a respective sample well 40 when the collection unit 100 is placed on the lower surface of the sample block 20.

In operation, the sample wells 40 contain an electrophoretic medium such as polyacrylamide gel. A biological sample is loaded onto the electrophoretic medium through the well openings 92 of the upper buffer chamber 10. Preferably, a single sample is loaded onto the upper surface of the electrophoretic medium in each sample well 40. Upper electrophoretic buffer is loaded into the upper buffer chamber 20, before or after the samples are loaded. Accordingly, the upper electrodes 90 are also submerged in the same buffer. In a preferred embodiment, buffer is circulated between the upper and lower buffer chambers. Circulation of buffer between the upper and lower buffer chambers increases the efficiency of separating and purifying molecular samples. Buffer circulation may be accomplished through the use of an accessory pump and a buffer transfer channel. Preferably, the buffer transfer channel is positioned so as to not distort the uniform electric field applied to the molecular samples.

Similarly, the bottom electrode unit 30 is submerged, in a lower buffer chamber, in a lower electrophoretic buffer that covers the lower electrode pins 70. The upper 90 and lower 70 electrodes are connected to a power source and an electric potential is applied across the sample wells 40. The samples migrate through the electrophoretic medium and the sample components are separated as a function of their size and charge. Preferred material for the electrodes includes platinum, copper, and aluminum, but any metallic substance may be used.

When a predetermined level of electrophoretic migration is reached, the potential across the electrodes 70 is removed and a collection unit 100 is connected to the lower surface of sample block 20 and a fraction of each sample is electrophoresed into a collection chamber 110 of the collection unit 100.

In a preferred embodiment, the sample block 20 is turned upside down with respect to the upper and lower buffer chambers and the collection unit 100 is placed on the lower surface of the sample block 20 (the lower surface is now facing up). The sample block 20 and associated collection unit 100 are reconnected to the power supply via electrophoretic buffer, preferably using the same upper and lower buffer chambers. To remove unwanted sample, fresh buffer is added to both the upper and lower buffer chambers before starting sample collection. The collection unit 100 is adapted to be placed on the sample block 20 when it is connected to the upper buffer chamber. In order to electrophorese a fraction of each sample into the collection unit 100 placed on top of the lower surface of the sample block 20, an electric field is reapplied with a polarity that is inverse with respect to the polarity that was originally used.

In an alternative embodiment, the sample block 20 is not turned upside down, and the collection unit 100 is associated with the lower surface of the sample block 20. The collection unit 100 is preferably attached to the sample block 20. Alternatively, the collection unit may be placed in the lower buffer chamber so that the lower surface of the sample block 20 rests on the upper surface of the collection unit 100. However, in general, the collection unit is adapted to be placed between the sample block 20 and the lower electrode in the lower buffer chamber. In this embodiment, an electric field is reapplied with the same polarity in order to electrophorese a fraction of each sample into the collection unit 100.

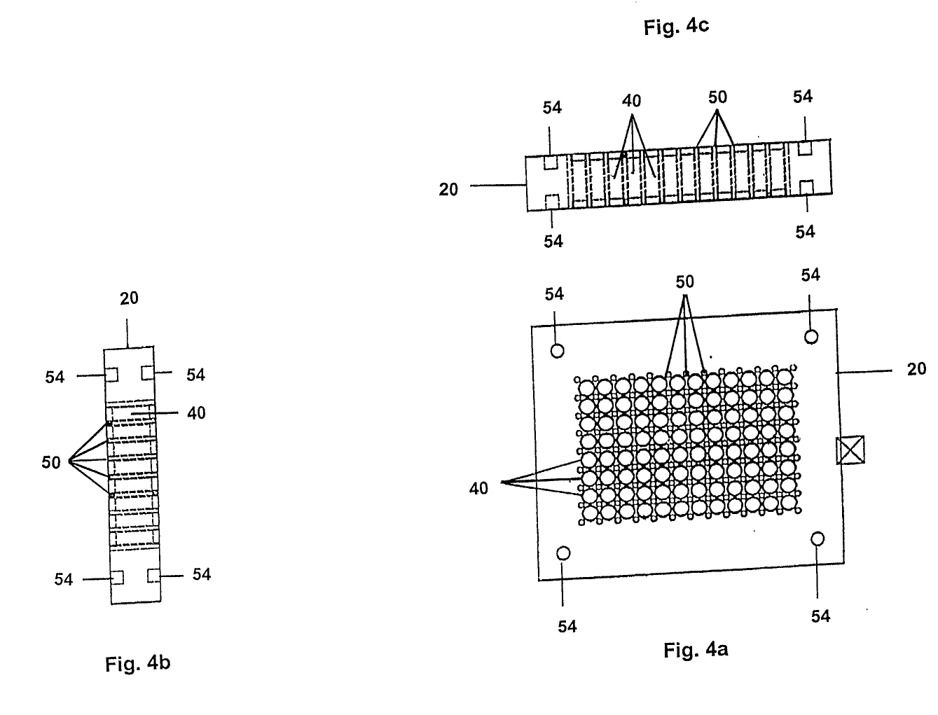

In more detail and referring also to FIGS. 2 and 4, the sample block 20 of the invention includes multiple wells 40. In one embodiment, the wells 40 comprise an array of cylindrical openings drilled into a block of electrically non-conductive material. The cross-section of the wells 40 need not be cylindrical but can be of any shape suitable for electophoresis. A preferred sample well diameter is 7 mm, but any size suitable for electophoresis may be used. Reducing the cross-sectional area of the sample column reduces the maximum amount of molecular sample that can be loaded and allows the sample array density to be increased.

The sample block 20 also can be any shape, provided it contains a two-dimensional array of wells 40. It is not required that the array of wells 40 be a regular array. In one embodiment, the sample block is configured of an 8 by 12 array of 96 wells. In a further embodiment, the sample block is a milled block. In one embodiment, the sample block is made of clear acrylic. Alternative materials for manufacturing the sample block and electrode units include injection molded plastic and glass. In preferred embodiments the upper and lower buffer chambers, sample block, electrodes, and collection units are discrete units that are manufactured independently. However, any combination of these units could be manufactured as a single piece. For example, the lower electrode could be manufactured as part of the lower buffer chamber. In one embodiment, an apparatus comprising the electrodes, the sample block, and the buffer chambers is made as a single device.

In a preferred embodiment of the invention, the sample block measures 12 cm in length and 8 cm in width. In a further preferred embodiment, a sample block height, or corresponding sample well length, of 3 cm is the preferred length for purifying DNA fragments in the size range sufficient for cloning into high-copy plasmid vectors. Shortening the sample well length results in decreased time to process biological samples and decreased precision in separating and purifying specific biological molecules from the samples. In a preferred sample block measuring 12 cm in length, 8 cm in width, and 3 cm in height, a current of 45 V and 250 mA is optimal for separation and purification of samples.

Interspersed with the wells 40 are a plurality of capillary gas removal passageways 50 which are positioned along side the wells 40 in the sample block 20 and remove gas generated during the electrophoretic process. The maximum diameter of the gas removal capillaries is limited by the space left between the sample columns. As gas accumulates at the electrode pins 70 in bottom electrode unit 30, the gas migrates through the capillary gas removal passageways 50 so as to keep the gas from interfering with the electrophoretic process by accumulating at the lower surface of the electrophetic medium in the wells 40. Preferably, the gas removal passageways 50 are aligned with the electrical elements of the bottom electrode unit 30. Accordingly, if the bottom electrode unit includes one or more wires that run along its surface, preferred gas removal passageways are grooves in the lower surface of the sample block 20 that are in the same general configuration as the electrode wires. The maximum width of these grooves is limited to the distance between rows or columns of the sample wells. Preferably, a gas removal groove along the lower surface of a sample block is connected to one or more sides of the sample block so that the groove is useful to direct gas away from the lower surface of the sample wells.

In alternative embodiments, capillary gas removal passageways may be used with a lower electrode that includes wires. Similarly, a sample block with grooves along its lower surface can be used in association with an electrode that includes a plurality of electrical pins. In a further embodiment, a sample block can include grooves on its lower surface and capillaries that connect its lower and upper surfaces. The capillaries are preferably connected to the grooves so that the grooves direct gas bubbles to the capillaries. In a further embodiment, gas removal passageways 50 extend above the upper surface of the housing and are high enough to extend above the surface of the upper electrophoretic buffer when the device is in operation. In an alternative embodiment, the lower surface of a sample block includes grooves, indents, or cavities that are large enough to contain all of the gas that is generated during a typical use of the electrophoretic device. In this embodiment, the gas is not removed from the lower surface of the sample block, but is directed into the grooves, indents, or cavities and away from the lower surface of the sample wells. Accordingly, the grooves, indents, or cavities are preferably located so that they are placed above the conducting elements of an electrode when the device is in operation.

Finally, in a preferred embodiment, the sample block 20 includes a series of registration holes 54 located on both surfaces of the block 20. The holes are positioned to accept registration plugs 56 located on one surface of both the upper buffer chamber 10 and the bottom electrode unit 30. The use of the plugs 56 and registration holes 54 assure that the sample well openings 92, the sample wells, the electrode pins 70 and the collection chambers are properly aligned when the upper buffer chamber 10, the sample block 20 and the bottom electrode unit 30 or collection unit 100 are assembled.

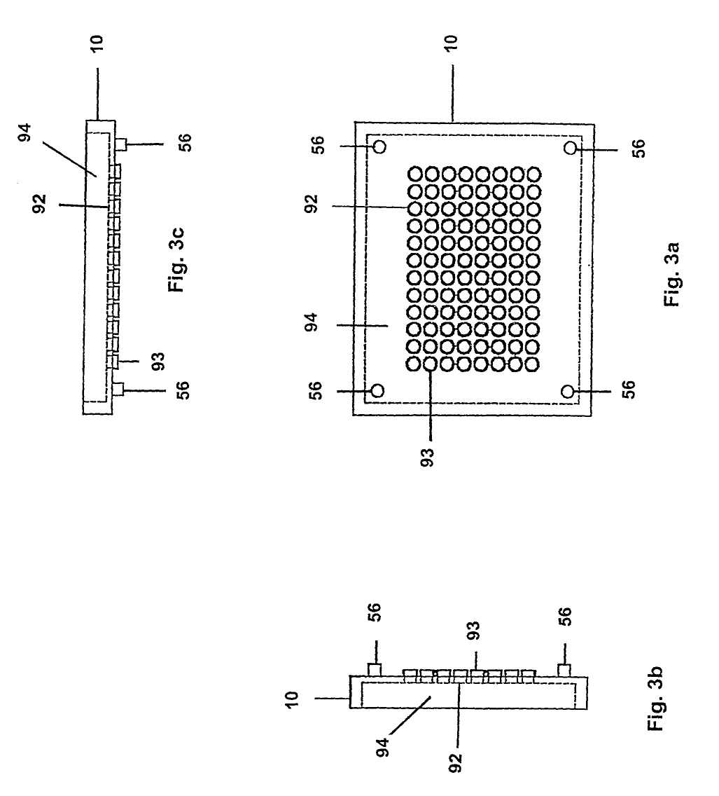

Referring also to FIG. 3, the upper buffer chamber 10 defines a reservoir 94 into which is placed the electrophoretic buffer. The surface of the reservoir 94 includes an array of sample well openings 92 each of which, in one embodiment, includes a tubular projection 93 which is positioned, sized and shaped to fit tightly within the sample wells 40 of the sample block 20. In this way, fluid from the reservoir 94 is directed into the sample wells 40 without leakage. As mentioned previously plugs 56 extending from the upper buffer chamber 10 are located and sized to fit in the registration holes 54 of the sample block 20 to align the upper buffer chamber 10 with the sample block 20 during assembly. In an alternative embodiment, an upper buffer chamber 10 is permanently attached to, or is a part of, the sample block 20.

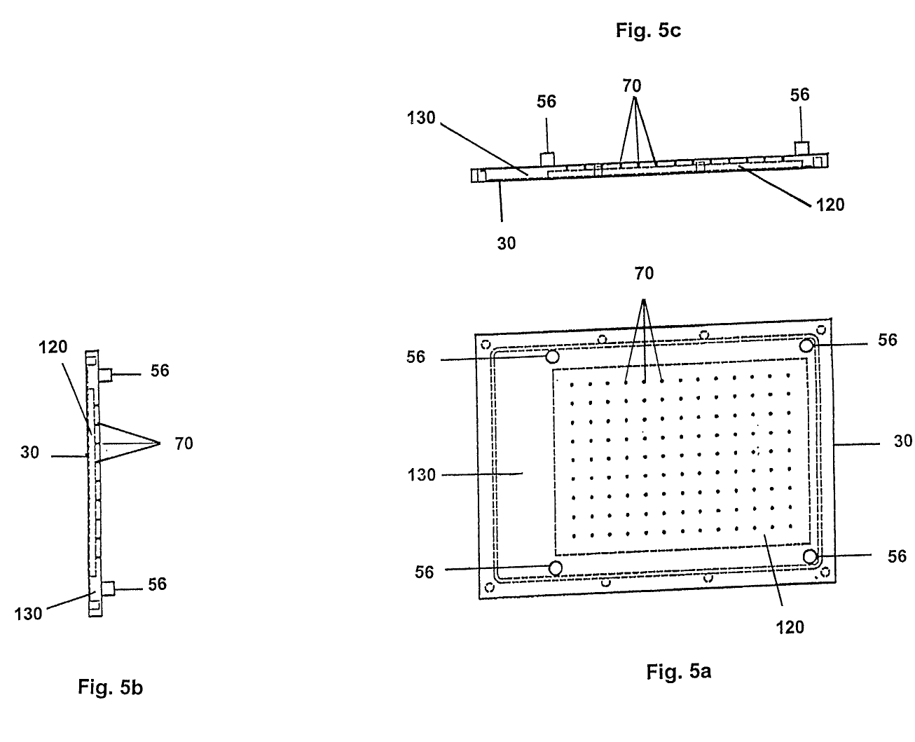

Referring also to FIG. 5, a preferred bottom electrode unit 30 includes an electrically conductive plate 120 located within an electrically insulating plate 130. The electrically insulating plate 130 includes openings for a plurality of electrode pins 70 which are electrically connected to the electrically conducting plate 120. Preferably, each of the pins 70 is disposed to be positioned below a respective one of the gas removable passageway of the sample block 20. In operation, the electrically conducting plate 120 is connected to a voltage source. In an alternative embodiment, there are no electrode pins 70 but instead a plurality of conductors, such as platinum wires, are distributed along the upper surface of the bottom electrode unit 30 and are connected to the voltage source. The electrical wire need only be connected at one point to each electrode unit, provided that the resistance of the electrode material across the cross-sectional dimension of the sample array is negligible.

An upper buffer chamber 10 can include one or more electrode wires or a plurality of electric pins connected to a conducting plate. The configuration of the upper electrode is not as important, because gas generated by electrolysis at the upper electrode rises to the surface of the upper electrophoretic buffer and does not disrupt the electric current running through the sample wells. However, a preferred electrode of the upper buffer chamber 10 in conjunction with the bottom electrode unit is adapted to generate a uniform current across a two-dimensional array of sample wells. By providing a uniform current or electric field throughout the plurality of wells, it is possible to efficiently, reproducibly, and simultaneously separate and isolate biological molecules of a chosen size from a large number of molecular samples. Uniformity of migration in the different wells allows for the collection of the same target molecules from each of the sample wells. Preferably, a uniform electric potential between the cathode and anode of the device across each sample is maintained by precise spacing, and the maintenance of uniform electrical conductivity for all materials involved, including the buffer.

In one embodiment, the collection unit 100 is placed on the lower surface of the sample block to collect a target portion of each sample when the molecular samples have reached a predetermined position in the medium during electrophoresis. A preferred collection unit 100 is adapted to fit on the lower surface of the sample block 20 and collect fractions from multiple sample wells 40. Preferably, a collection unit 100 includes individual sample collection elements to minimize cross-contamination of the collected fractions. For example, target samples can be collected by attaching a filter plate. In a preferred embodiment, the collection unit 100 is comprised of a membrane that binds the target biological molecule. Preferably, the membrane allows current to flow through it. Collection units can include several types of filtration materials, thereby making the device practical for several purification processes. In one embodiment, a positively charged filter plate is used to collect nucleic acids. The filtration materials may operate by other molecular binding principles, such as steptavidin-biotin or antibody biding. The filtration material may also work by size exclusion. One example of a size exclusion filtration material is a dialysis membrane.

In general, a preferred embodiment of the invention is disposable. In a more preferred embodiment, a sample block is provided with electrophoretic medium in each sample well. A sample block including electrophoretic medium may be provided by itself or in combination with one or more additional components of the electrophoretic apparatus (e.g., an electrode, buffer chamber, or sample collection unit).

Methods of the invention are useful to collect different fractions of molecular samples. Methods and devices of the invention can be used to separate samples within a 500 base pair size range. However, one of ordinary skill in the art will appreciate that any size range can be collected by optimizing the properties of the electrophoretic medium, such as the concentration of agarose or acrylamide, according to known separation principles. In addition, different sample well sizes can be used. For example, the length of the sample wells can be varied by varying the distance between the upper and lower surfaces of the sample block. The length of the sample wells affects the separation properties of the device according to known separation principles. The diameter of a sample well can also be varied in order to vary the amount of sample that can be separated in the sample well.

In a further embodiment, multiple sample fractions can be collected from a single sample. For example, a first collection unit 100 is attached to the electrophoretic medium to collect a first target portion of a sample. The first collection unit 100 is removed, and a second collection unit 100 subsequently is attached to collect a second target portion of the sample. In general, the size range of the sample components that are collected in a sample fraction is a function of the physical properties of the electrophoretic medium, and the electrophoretic time and voltage used during the elution into the collection unit 100. For example, applying 45 volts for one hour using 0.8% agarose in 1X TAE buffer results in size ranges averaging 1.5 kb being collected.

Methods of this invention have the advantage of not requiring visualization of the sample being purified. In a preferred embodiment, a tracking agent is used to monitor the position of the molecular sample as it migrates through the medium. Known DNA size standards or colored dyes, such as BlueView™ or ChromaTrack™ (Sigma-Aldrich, St. Louis, Mo.), can be used to track the sample as it migrates through the electrophoretic medium. In a preferred embodiment, tracking agent is added to a single sample well and used to track the migration of samples in other wells of the sample block. When the tracking agent reaches a predetermined position in its sample well, for example the bottom of the agarose or polyacrylamide column, the electrophoretic current is reduced or removed and a collection plate is attached as described above. Current is reapplied and target molecules are collected in the collection unit.

The following example provides further details of the devices and methods according to the invention. For purposes of exemplification, the following example provides details of the use of the present invention with a 96-well device for collection of DNA fragments. Accordingly, while exemplified in the following manner, the invention is not so limited and the skilled artisan will appreciate its wide range of application upon consideration thereof.

### EXAMPLES

### Example 1

The device and method of the invention are useful for collecting DNA fragments. In this example, DNA fragments measuring 1.75 kb in length were collected. The electrophoretic medium used was agarose. Agarose was prepared by mixing 3 g of agarose in 275 ml of water in a flask and heating the flask for 7 minutes. The hot agarose mixture was poured into a 500 ml graduated cylinder and 30 ml of 10X TAE BlueView™ colored dye was pipetted into the cylinder. Water was added to the cylinder to bring the volume up to 300 ml, with the final concentration of the mixture being 1% agarose. The agarose was then poured back in the flask and placed in water bath at 55° C. to cool.

A milled, clear, acrylic block containing 96 tubular columns and 117 capillaries drilled through the block was covered on one side of the block with adhesive tape. The block was covered and placed in a tray with the tape side down. The prepared agarose mixture was poured into the open side of the block. After the agarose was left to solidify for 30 minutes, the agarose was leveled off on the top of the block using a razor blade. The tape was then peeled off the bottom of the block. The agarose from the 117 capillaries was removed by using a 117 prong ramrod to push the agarose out of the capillaries. Agarose was also removed from two of the tubular columns to allow buffer to circulate through.

Running buffer was prepared by combining 150 ml of 10X TAE BlueView™ with 1350 ml of water. The buffer (1X TAE BlueView™) was placed under a vacuum for 15 min to remove excess gas. The buffer was poured into the bottom electrode until it was one inch from the top. The milled acrylic block containing the agarose filled columns was placed on top of the bottom electrode.

A 96-well loading plate was clamped onto the top electrode chamber. The top electrode assembly was placed on top of the milled acrylic block, so that each loading plate well inserts into its respective agarose column. The top electrode assembly was pushed down and buffer was allowed to rise into the upper buffer chamber. The remainder of the buffer was added to the top electrode chamber. A pipette was used to remove trapped air in the plate wells.

A size standard was prepared consisting of 60 μl of Bgl digested pUC19 (250 ng/μl), 10 μl of 50% glycerol, 10 μl of 5% Blue dextran, and 20 μl of water. 50 μl of the size standard was loaded in each of two wells. 12.5 μg of sample was loaded per column using a multi-channel pipette. Electrodes were plugged into a 45 V power supply. After 45 minutes, the sample was near the bottom of the agarose column. The electrodes were disconnected from the power supply when the last of the two blue DNA bands in the size standard reached the bottom and started to run off the column. Buffer was poured out of the device. The assembled device was filled with water and the water was drained. This wash was repeated again to remove all unwanted fragments.

To collect the desired DNA fragments, the bottom plastic mesh of a 96-well positively-charged diethylaminoethyl (DEAE) cellulose filter plate (Millipore, Bedford, Mass.) was pulled off. With a razor, the filter material was removed from two wells of the plate for buffer circulation. The empty plate wells aligned with the empty agarose columns. Using a multi-channel pipette, 250 μl of 1X TAE buffer was added to the DEAE plate to remove air from the plate. The DEAE plate was clamped onto the upper electrode, replacing the loading plate. The bottom electrode was filled with buffer again one inch from the top. The milled acrylic block was again placed on the bottom electrode, but with the desired DNA at the top instead of the bottom. The DEAE plate/electrode assembly was placed on to the milled acrylic block in the same fashion as the loading plate. DNA fragments measuring 1.75 kb in length were collected using a power supply timer of 45 V for 7 minutes with the polarity of the electrodes reversed. Accordingly, the DNA migrated to the top of the device.

For final purification of the sample, wash buffer consisting of 0.1 M NaCl and 10 mM Tris with a pH of 8.0 was prepared. Also, elution buffer consisting of 1 M NaCl and 10 mM Tris with a pH of 8.0 was prepared. The plastic mesh that had been removed was snapped back onto the DEAE plate. The DEAE plate was placed in a vacuum manifold to vacuum out the running buffer. The samples were washed twice with 200 μl of wash buffer. After all the wash was gone, 100 μl of elution buffer was added to each well. After 1 minute, the elution solution was vacuumed into a 96-well plate with a U-shaped bottom. 10 μl of magnetic carboxyl-coated beads (Seradyn, Indianapolis, Ind.) and 77 μl of isopropanol was then added to each sample well. The solution was mixed and the DNA was allowed to bind to the beads. The plate containing the beads and DNA was placed on a magnet plate for 15 minutes. The supernatant was then pipetted off. The beads were washed once with 200 μl of 80% ethanol while still on the magnet plate. The wash was removed and the plate was left to dry. The sample was then eluted with 25 ul of 10 mM Tris with a pH of 8.0.

While the invention has been shown and described with reference to specific preferred embodiments, it should be understood by those skilled in the art that various changes in form and detail may be made therein without departing from the spirit and scope of the invention as defined by the following claims.

What is claimed is:

1. A device for simultaneous electrophoresis of a plurality of molecular samples, the device comprising:
(a) a housing comprising an upper surface and a lower surface;
(b) a two-dimensional array of a plurality of sample wells, wherein each sample well is in fluidic communication with both said upper surface and said lower surface; and
(c) a gas removal passageway on said lower surface, wherein said passageway is in fluidic communication with said lower surface.

2. The device of claim 1, further comprising a collection unit that attaches to the said array of sample wells.

3. The device of claim 1, wherein said collection unit comprises a two-dimensional-array of collection elements.

4. The device of claim 2, wherein each collection element comprises a positively charged diethylaminoethyl (DEAE) cellulose filter plate.

5. The device of claim 1, wherein said passageway extends across the lower surface of the housing.

6. The device of claim 1, wherein said passageway connects the lower surface of the housing to the upper surface of the housing.

7. The device of claim 1 comprising a plurality of gas removal passageways, wherein each passageway is in fluidic communication with the lower surface of the housing.

8. The device of claim 1, wherein said sample wells comprise an electrophoretic medium.

9. The device of claim 8, wherein said electrophoretic medium comprises agarose.

10. The device of claim 8, wherein said electrophoretic medium comprises polyacrylamide.

11. The device of claim 1, wherein said housing is an acrylic block.

12. An apparatus for simultaneous separation and purification of a plurality of molecular samples by electrophoresis, the apparatus comprising:
(a) a housing having an upper surface and a lower surface and comprising a plurality of sample wells in fluidic communication with said upper surface and said lower surface;
(b) a gas removal passageway on said lower surface; and
(c) an electrode located substantially under said passageway.

13. The apparatus of claim 12, wherein said electrode comprises:
(a) an electrically conducting plate having an upper and a lower surface; and
(b) an electrically insulating plate having an upper and a lower surface and comprising a plurality of electrode pins, wherein the lower surface of said insulating plate is attached to the upper surface of said conducting plate, and wherein each electrode pin is in electrical connection with said conducting plate and with the upper surface of said insulating plate.

14. The apparatus of claim 12, wherein said housing is removably attached to said electrode.

15. The apparatus of claim 12, wherein said housing is adapted to receive a collection unit.

16. The apparatus of claim 12, wherein said passageway extends across the lower surface of the housing.

17. The apparatus of claim 12, wherein said passageway connects the lower surface of the housing to the upper surface of the housing.

18. The apparatus of claim 12, comprising a plurality of gas removal passageways, wherein each passageway is in fluidic communication with the lower surface of the housing.

19. The apparatus of claim 12, further comprising a collection unit that attaches to the said array of sample wells.

20. The apparatus of claim 12, wherein said collection unit comprises a two-dimensional-array of collection elements.

21. The apparatus of claim 20, wherein each collection element comprises a positively charged diethylaminoethyl (DEAE) cellulose filter plate.

22. An electrode for generating a uniform current across an array of electrophoretic sample wells, said electrode comprising:
(a) an electrically conducting plate having an upper and a lower surface; and
(b) an electrically insulating plate having an upper and a lower surface and comprising a plurality of electrode pins, wherein the lower surface of said insulating plate is attached to the upper surface of said conducting plate, and wherein each electrode pin is in electrical connection with said conducting plate and with the upper surface of said insulating plate.

23. The electrode of claim 22, wherein said electrode pins are platinum pins.

24. The electrode of claim 22, comprising a two-dimensional array of electrode pins.

25. A method for simultaneous separation and purification of a molecular sample by electrophoresis, the method comprising the steps of:
(a) loading a molecular sample onto an electrophoretic medium;
(b) applying an electrical field to the medium to induce the molecular sample to migrate through the medium; and
(c) attaching a collection unit to the medium when the molecular sample reaches a predetermined position in the medium.

26. The method of claim 25, further comprising the steps of:
(d) reducing the electrical field before said collection unit is attached to the medium; and
(e) applying an electrical field to the medium to induce the molecular sample to migrate to the collection unit after said collection unit is attached to the medium.

27. The method of claim 26, wherein said electrical field is entirely removed in step d).

28. The method of claim 25, wherein said collection unit comprises a positively charged diethylaminoethyl (DEAE) cellulose filter plate.

29. The method of claim 25, wherein said collection unit is a first collection unit, further comprising the steps of:
(d) attaching a second collection unit; and
(e) applying an electrical field to induce a second portion of the molecular sample to migrate to the second collection unit.

30. The method of claim 29, further comprising repeating steps d) and e).

31. The method of claim 25, wherein a tracking agent is used to monitor the migration of the molecular sample in the electrophoretic medium.

32. The method of claim 31, wherein said tracking agent comprises a known nucleic acid size standard.

33. The method of claim 31, wherein said tracking agent comprises a colored dye.

34. The method of claim 25, wherein said molecular sample comprises a nucleic acid.

35. The method of claim 25, wherein said molecular sample comprises a peptide.

36. The method of claim 25, wherein said electrophoretic medium comprises an agarose gel.

37. The method of claim 25, wherein said electrophoretic medium comprises a polyacrylamide gel.

38. A method for simultaneous separation of a plurality of molecular samples, the method comprising the steps of:
(a) loading a plurality of molecular samples onto a two-dimensional array of sample wells, each well containing an electrophoretic medium; and
(b) applying an electrical field to each sample well, thereby to separate said plurality of molecular samples.

39. The method of claim 38, further comprising providing one or more passageways to remove gas produced by one or more electrodes immersed in aqueous solution.

40. The method of claim 39, wherein said passageways are longitudinally aligned with said electrodes.

41. The method of claim 39, wherein said passageways are transversely aligned with said electrodes.

42. The method of claim 38, wherein said electric field is substantially uniform across said plurality of wells.

43. The method of claim 38, wherein said two-dimensional array comprises wells that are arranged adjacent to each other in a generally longitudinal orientation.

44. The method of claim 38, wherein said molecular samples contain nucleic acids.

45. The method of claim 38, wherein said molecular samples contain peptides.

46. The method of claim 38, wherein said electrophoretic medium comprises an agarose gel.

47. The method of claim 38, wherein said electrophoretic medium comprises a polyacrylamide gel.

48. A method for simultaneous separation and purification of a plurality of molecular samples by electrophoresis, the method comprising the steps of:
(a) loading a plurality of molecular samples onto a two-dimensional array of sample wells, each well containing an electrophoretic medium;
(b) applying an electrical field to said sample wells to induce the molecular samples to migrate through the electrophoretic medium; and
(c) attaching a collection unit to said array of sample wells when said molecular samples reach a predetermined position in said wells.

49. The method of claim 48, further comprising the steps of:
(d) reducing the electrical field before said collection unit is attached to the medium; and
(e) applying an electrical field to said wells to induce the molecular samples to migrate to said collection unit after the collection unit is attached to the medium.

50. The method of claim 49, wherein said electrical field is entirely removed in step d).

51. The method of claim 48, wherein said electric field is substantially uniform across said plurality of wells.

52. The method of claim 48, further comprising providing one or more passageways to remove gas produced by one or more electrodes immersed in aqueous solution.

53. The method of claim 52, wherein said passageways are longitudinally aligned with said electrodes.

54. The method of claim 52, wherein said passageways are transversely aligned with said electrodes.

55. The method of claim 48, wherein said collection unit comprises a two-dimensional-array of collection elements.

56. The method of claim 55, wherein each collection element comprises a positively charged diethylaminoethyl (DEAE) cellulose filter plate.

57. The method of claim 48, wherein said molecular samples contain nucleic acids.

58. The method of claim 48, wherein said molecular samples contain peptides.

59. The method of claim 48, wherein said electrophoretic medium comprises an agarose gel.

60. The method of claim 48, wherein said electrophoretic medium comprises a polyacrylamide gel.

61. An apparatus for simultaneous separation and purification of a plurality of molecular samples by electrophoresis, the apparatus comprising:
(a) means for housing a plurality of sample wells, said housing having a surface; and
(b) means for removing gas produced by an electrode at the housing surface to prevent the gas from interfering with the electrophoresis in the plurality of sample wells.

62. The apparatus of claim 61, further comprising a means for collecting sample fractions from said wells.
